(19) United States
(12) Patent Application Publication
(10) Pub. No.: US 2008/0026044 A1
(43) Pub. Date: Jan. 31, 2008

(54) Combination Chemotherapy Comprising Capecitabine and a Liposomal Platinum Complex

(76) Inventors: Jonathan Lewis, Fairfield, CT (US); Axel Hoos, New Haven, CT (US); Robert Peter Gale, Los Angeles, CA (US)

Correspondence Address: JONES DAY, 222 EAST 41ST ST, NEW YORK, NY, 10017, US

(21) Appl. No.: 10/557,672

(22) PCT Filed: May 20, 2003
(86) PCT No.: PCT/US03/15770

§ 371 Date: Apr. 11, 2007

(51) Int. Cl. A61K 31/7028; A61K 9/127; A61P 35/00

(52) U.S. Cl. 424/450; 514/49

(57) ABSTRACT

The present invention relates to methods for treating cancer comprising administering a combination of capecitabine and a liposomal platinum complex, pharmaceutical compositions comprising capecitabine and a liposomal platinum complex, and kits comprising unit dosage forms of capecitabine and a liposomal platinum complex.

### 1. FIELD OF THE INVENTION

The present invention relates to combination therapies comprising capecitabine and a liposomal platinum complex, pharmaceutical compositions comprising capecitabine and a liposomal platinum complex, and methods for treating cancer comprising administering a combination of capecitabine and a liposomal platinum complex.

### 2. BACKGROUND OF THE INVENTION

Cancer is second only to cardiovascular disease as a cause of death in the United States. The American Cancer Society estimated that in 2002, there were 1.3 million new cases of cancer and 555,000 cancer-related deaths. There are currently over 9 million living Americans who have been diagnosed with cancer and the NIH estimates the direct medical costs of cancer as over $100 billion per year with an additional $100 billion in indirect costs due to lost productivity—the largest such costs of any major disease.

Modalities useful in the treatment of cancer include chemotherapy, radiation therapy, surgery and biological therapy (a broad category that includes gene-, protein- or cell-based treatments and immunotherapy). See, for example, Stockdale, “Principles of Cancer Subject Management”, in Scientific American Medicine, vol. 3, Rubenstein and Federman, eds., (1998), Chapter 12, Section IV.

Despite the availability to the clinician of a variety of anticancer agents, traditional chemotherapy has many drawbacks. See, for example, Stockdale, 1998, “Principles Of Cancer Subject Management” in Scientific American Medicine, vol. 3, Rubenstein and Federman, eds., (1998), Chapter 12, Section X. Almost all anticancer agents are toxic, and chemotherapy can cause significant, and often dangerous, side effects, including severe nausea, bone marrow depression, liver, heart and kidney damage, and immunosuppression. Additionally, many tumor cells eventually develop multi-drug resistance after being exposed to one or more anticancer agents. As such, single-agent chemotherapy can cure only a very limited number of cancers. Most chemotherapeutic drugs act as anti-proliferative agents, acting at different stages of the cell cycle. Since it is difficult to predict the pattern of sensitivity of a neoplastic cell population, or the current stage of the cell cycle that a cell happens to be in, it is common to use multi-drug regimens in the treatment of cancer.

The basic principles of combination chemotherapy involve the selection of agents that: (i) have proven to be active against the specific cancer being treated; (ii) have different mechanisms of action or which act at different stages of the cell cycle; and (iii) have non-overlapping toxicities. Multidrug regimens have resulted in significant increases in cure rates and in overall survival in a large number of cancers compared with single-drug regimens. Cancers that may be cured with administration of combination chemotherapy alone, include Burkitt's lymphoma, choriocarcinoma, acute leukemia, bladder and testicular cancer, Hodgkin's disease, testicular cancer, small cell lung cancer, and nasopharyngeal cancer.

Thus, there is a significant need in the art for novel compounds, compositions, and methods that are useful for treating cancer with improved therapeutic indices.

Capecitabine (5′-deoxy-5-fluoro-N-[(pentyloxy)carboyl]-cytidine) is a fluoropyidine carbamate analog with antitumor activity. Capecitabine is used as monotherapy and in combination therpy regimens for the treatment and palliative management of various forms of cancer including colorectal and breast cancer. Despite its demonstrated clinical usefulness, there are a number of serious disadvantages associated with the use of capecitabine which can be dose-limiting and which may render patients unable to tolerate treatment using capecitabine. Adverse reactions commonly seen during systemic therapy using capecitabine, include diarrhea, stomatitis, nausea and vomiting, hand-and-foot syndrome, anemia, hyperbilirubinemia, dermatitis and alopecia Other adverse effects associated with the systemic administration of capecitabine include constipation, abdominal pain, edema, decrease appetite, dyspnea, back pain, neutropenia, nail disorders, pyrexia, asthenia, fatigue, weakness, headache dizziness, anorexia, arthralgia, myaligia, neutropenic fever, cough, sore throat, leukopenia and thrombocytopenia.

Platinum coordination complexes were first identified as cytotoxic agents in 1965. cis-diamminedichloroplatinum (cisplatin) is a clinically significant anticancer agent useful for the treatment of a broad spectrum of neoplastic diseases in humans. Loehrer et al., Ann. Int. Med. 1984, 100:704-713. However, long-term administration of cisplatin is limited by severe systemic toxicity, including emesis, nephrotoxicity, ototoxicity and neurotoxicity. Zwelling et al., “Platinum Complexes” in Pharmacologic Principles of Cancer Treatment, Ed. B. A. Chabner, Saunders, Philadelphia, Pa. (1982). cis-diamiine(1,1-cyclobutanedicarboxylato) platinum (carboplatin), is a second-generation platinum analog and is the only platinum drug other than cisplatin to enjoy widespread use in the clinic. Carboplatin is effective when used in place of cisplatin in established chemotherapeutic drug regimens and although less emetic, nephrotoxic, neurotoxic, and ototoxic than cisplatin, carboplatin has undesirable myelosuppressive properties that cisplatin does not. Go et al., J. Clin. Oncol. 1999, 17(1): 409-22. Oxaliplatin is a recently developed third-generation cisplatin analog with an 1,2-diaminocyclohexane (DACH) carrier ligand which has displayed clinical activity in a variety of tumor types and is not cross-resistant with cisplatin and carboplatin. Oxaliplatin is reported to act synergistically with gemcitabine in both gemecitabine resistant and chemotherapy-naive disease and is currently being evaluated as a single-agent and in combination regiments against breast, lung, prostate and germ cell cancers, malignant mesothelioma, and non-Hodgkin's lymphoma. Misset et al., Crit Rev. Oncol. Hematol. 2000, 35(2): 75-93.

L-NDDP is a liposomal formulation of the platinum complex cis-bis-neodecanoato-trans-R,R-1,2-diaminocyclohexane, and is currently showing promise in clinical trials for pancreatic cancer, metastatic colorectal cancer and malignant mesothelioma. It is speculated that bis-neodecanoato-cis-1,2-diaminocyclohexane platinum (II) (NDDP) undergoes an intraliposomal chemical transformation to provide an active platinum species. Perez-Soler et al., Cancer Chemother. Pharmacol. 1994, 33:378-384.

Despite the significant research efforts and resources which have been directed towards the development of novel anticancer agents and improved methods for treating cancer there is a significant need in the art for treatment regimens with improved therapeutic indices that are useful for treating cancer.

The recitation of any reference in this application is not an admission that the reference is prior art to this application.

### 3. SUMMARY OF THE INVENTION

The present invention relates to a combination of anticancer agents, and to methods for treating cancer comprising administering the anticancer agents to a subject in need thereof.

Accordingly, in one aspect, the invention provides a method for treating cancer, said method comprising:

(a) administering to a subject in need thereof an amount of L-NDDP; and

(b) administering to said subject an amount of capecitabine or a pharmaceutically acceptable salt thereof.

In a specific embodiment, the amounts administered are together effective to treat cancer.

In one embodiment, capecitabine or a pharmaceutically acceptable salt thereof, is administered prior to the administration of the liposomal platinum complex.

In another embodiment, capecitabine or a pharmaceutically acceptable salt thereof, is administered concurrently with the liposomal platinum complex.

In still another embodiment, capecitabine or a pharmaceutically acceptable salt thereof, is administered subsequent to the administration of the liposomal platinum complex.

In another aspect, the invention provides a method for treating cancer, said method comprising:

(a) administering to a subject in need thereof capecitabine or a pharmaceutically acceptable salt thereof; and

(b) administering to said subject a platinum complex having the formula

DACH-Pt-X₂

wherein said platinum complex is entrapped in a liposome, and where DACH is diaminocyclohexane and X is -halogen or a lipid ligand.

In still another aspect, the invention provides a method for treating cancer, said method comprising:

(a) administering to a subject in need thereof capecitabine or a pharmaceutically acceptable salt thereof; and

(b) administering to said subject a platinum complex having the formula

DACH-Pt-Cl₂

wherein said platinum complex is entrapped in a liposome, and where DACH is diaminocyclohexane.

In a further aspect, the invention provides a method for treating cancer, said method comprising:

(a) administering to a subject in need thereof capecitabine or a pharmaceutically acceptable salt thereof; and

(b) administering to said subject a liposomal platinum complex, said liposomal platinum complex formed by a second method, said second method comprising making the pH of a composition comprising L-NDDP be acidic.

In yet another aspect, the invention provides a method for treating cancer, said method comprising:

(a) administering to a subject in need thereof capecitabine or a pharmaceutically acceptable thereof; and

(b) administering to said subject a liposomal platinum complex, said liposomal platinum complex formed by a second method, said second method comprising the steps:
(i) making the pH of a composition comprising L-NDDP be acidic; and
(ii) after a predetermined time, adjusting the acidic pH of the composition of step (i) to a pH greater than 7.

In a further aspect, the invention provides a method for treating cancer, said method comprising:

a) administering to a subject in need thereof an amount of a first pharmaceutical composition comprising L-NDDP or a degradation product thereof and a pharmaceutically acceptable carrier or diluent; and

(b) administering to said subject an amount of a second pharmaceutical composition comprising capecitabine or a pharmaceutically acceptable salt thereof, and a pharmaceutically acceptable carrier or diluent.

In a specific embodiment, the amounts administered are together effective to treat cancer.

The present invention also provides kits comprising a first container containing a unit dosage form of capecitabine or a pharmaceutically acceptable salt thereof, and a second container containing a unit dosage form of a liposomal platinum complex.

The details of the invention are set forth in the accompanying description below. Although any methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents, patent applications and publications cited in this specification are incorporated herein by reference for all purposes.

### 4. ABBREVIATIONS

The following abbreviations and their definitions, unless defined otherwise, are used in this specification: DACH is 1,2-diaminocyclohexane, DMSO is N,N-dimethylformamide, NDDP is cis-bis-neodecanoato-trans-R,R-1,2-diaminocyclohexane, and L-NDDP refers to a liposomal composition comprising NDDP.

### 5. DETAILED DESCRIPTION OF THE INVENTION

The anticancer agents to be utilized in the methods and compositions of the present invention can be administered in doses commonly employed clinically when such compounds are administered as monotherapy for the treatment of cancer. The anticancer agents can also act synergistically and in such cases can be administered in doses less than those commonly employed clinically when such compounds are administered as monotherapy for the treatment of cancer.

### 5.1 Liposomal Platinum Complexes

Liposomal platinum complexes useful in the invention include L-NDDP, which is a liposomal formulation of cis-bis-neodecanoato-trans-R,R-1,2-dicyclohexane platinum (II) (“NDDP”). Other liposomal platinum complexes useful in the invention include the liposomally encapsulated platinum complexes which result when the NDDP complex of L-NDDP undergoes an intraliposomal degradation reaction under acidic conditions, as described herein below.

L-NDDP is currently being evaluated in the clinic as a single-agent therapy for metastatic colorectal cancer and in combination therapy regimens for the treatment of colorectal cancer and pancreatic cancer.

Without being bound by theory, in one embodiment, a liposomal platinum complex of the invention can enter a cell by diffusion and react with DNA to form interstrand and intrastrand cross-links and DNA-protein crosslinks, which can interfere with the ability of the cell to replicate.

L-NDDP comprises NDDP, and a liposome comprising one or more liposomal lipid components. L-NDDP is typically prepared as a sterile, preliposomal lyophilate (i.e. does not contain liposomes at the time of lyophilization), said lyophilate comprising NDDP and one or more liposomal lipid components. Upon reconstitution in acidic solution, the preliposomal lyophilate forms a liposomal suspension of NDDP which is administered to a subject in need thereof. In a preferred embodiment, the liposomal product is formulated by reconstituting the preliposomal lyophilate using an acidified aqueous sodium chloride solution.

In one embodiment, L-NDDP is administered intravenously, intrapleurally, intra-arterially or intraperitoneally. In a preferred embodiment, L-NDDP is administered intravenously.

Methods of preparing NDDP and L-NDDP are well-known in the art, and are described, for example in U.S. Pat. No. 5,178,876 to Khokhar et al., which is incorporated herein by reference in its entirety. A procedure useful for preparing L-NDDP is presented in the examples section below.

When L-NDDP is exposed to an acidic environment, the liposomally entrapped NDDP complex can is converted via an acid-catalyzed degradation process to other platinum complexes which may possess anticancer activity. In one embodiment, L-NDDP is exposed to an acidic environment by reconstituting in an acidic solution, a preliposomal lyophilate comprising NDDP and a liposomal lipid component. In one embodiment, NDDP is entrapped in a liposome prior to exposing L-NDDP to acidic conditions. In one embodiment, NDDP is entrapped in a liposome prior to exposing L-NDDP to acidic conditions. In another embodiment, the entrapping of NDDP in a liposome is done in the presence of sodium chloride. In yet another embodiment, the entrapping of NDDP in a liposome is done in the presence of chloroform. In a specific embodiment, NDDP is entrapped in a liposome in the presence of chloroform via the preparation of L-NDDP by a method, said method comprising: (a) preparing a chloroform solution of NDDP and one or more liposomal lipid components; (b) concentrating said chloroform solution in vacuo so that a thin film results; (c) dispersing said thin film in aqeous sodium chloride to provide a suspension; (d) centrifuging said suspension to provide a solid residue; and (e) reconstituting said solid residue in an appropriate reconstitution media to provide L-NDDP. When using said method, residual chloroform can be present after said concentrating of step (b), and if so, will remain present up to and including reconstitution step (e) in which NDDP will be entrapped in a liposome in the presence of chloroform.

In another embodiment, L-NDDP can be exposed to an acidic environment when the liposome of L-NDDP comprises liposomal lipid components which are acidic (such as dimyristoyl phosphatidyl glycerol or dioleyl phosphatidyl glycerol).

The liposomal composition that results when L-NDDP decomposes upon exposure to an acidic environment may comprise more than one platinum complex, including but not limited to NDDP and complexes having the general formula

DACH-Pt-X₂

wherein each X independently includes, but is not limited to, halogen or a lipid ligand, wherein halogen is selected from —F, —Cl, —Br or —I, and the the lipid ligand(s) are derived from the liposomal lipids component(s) of the liposome. In a preferred embodiment, each occurrence of X is —Cl. For ease of reference, the term “liposomal platinum complex” as used herein will be understood to refer to both L-NDDP and to the liposomally encapsulated platinum complex(es) which result when either: (a) the pH of a composition containing L-NDDP is adjusted so that the pH is made acidic or (b) L-NDDP comprises a lipid ligand component which is an acidic lipid. In one embodiment, NDDP is entrapped in a liposome prior to the acidification. In a specific embodiment, the entrapping of NDDP in a liposome is done in the presence of sodium chloride or chloroform.

In one embodiment, L-NDDP comprises a liposomal lipid component which is an acidic lipid, preferably DMPG.

In another embodiment, the pH of a composition containing L-NDDP is made acidic by exposing L-NDDP to an acidic solution.

In another embodiment, the pH of a composition containing L-NDDP is made acidic by exposing L-NDDP to an acidic aqueous solution.

In still another embodiment, the pH of a composition containing L-NDDP is made acidic by exposing L-NDDP to an acidic aqueous sodium chloride solution.

In one embodiment, the pH of a composition containing L-NDDP is adjusted to a pH between 2.0 and 6.5.

In a specific embodiment, the pH of a composition containing L-NDDP is made acidic by reconstituting a preliposomal lyophilate comprising NDDP and a liposomal lipid component in an acidic saline solution, wherein said lyophilate does not contain liposomes at the time of lyophilization. In a preferred embodiment, the acidic saline solution has a pH of 3.

In one embodiment, a liposomal platinum complex comprises a platinum complex having the formula

DACH-Pt-X₂

entrapped in a liposome, where DACH is diaminocyclohexane and each X is independently -halogen or a lipid ligand.

In a specific embodiment, a liposomal platinum complex comprises a platinum complex having the formula

DACH-Pt-Cl₂

entrapped in a liposome, where DACH is diaminocyclohexane.

In another embodiment, the liposomal platinum complex is formed by a method, said method comprising adjusting the pH of a composition containing L-NDDP, so that the pH is made acidic.

In still another embodiment, the liposomal platinum complex is formed by a method, said method comprising adjusting the pH of a composition containing L-NDDP, so that the pH is made acidic, said platinum complex having the formula

DACH-Pt-X₂

where DACH is 1,2-diaminocyclohexane and each X is independently-halogen or a lipid ligand.

In still another embodiment, the liposomal platinum complex is formed by a method, said method comprising adjusting the pH of a composition containing L-NDDP in the presence of sodium chloride, so that the pH is made acidic, said platinum complex having the formula

DACH-Pt-Cl₂

where DACH is 1,2-diaminocyclohexane.

In a specific embodiment, the acid-catalyzed degradation of L-NDDP may be stopped after a predetermined time by adjusting the pH of an acidic L-NDDP formulation, said adjusting comprising adding to the acidic L-NDDP formulation an amount of a basic solution so that the resulting solution has a pH greater than 7.0.

In a specific embodiment the basic solution is a buffer solution.

In a preferred embodiment, the basic solution is phosphate buffered saline.

In one embodiment, the basic solution is added at time from about 0.5 hours to about 8 hours after the preliposomal lyophilate of L-NDDP is reconstituted in an acidic solution. In another embodiment, the basic solution is added at time from about 2 hours to about 6 hours after the preliposomal lyophilate of L-NDDP is reconstituted in an acidic solution.

Thus, in a specific embodiment, the liposomal platinum complex is formed by a method, said method comprising the steps:
(a) adjusting the pH of a composition comprising L-NDDP, so that the pH is made acidic; and
(b) after a predetermined time, adjusting the acidic pH of the composition of step (a) to a pH greater than 7.

In a further embodiment, the liposomal platinum complex is formed by a method, said method comprising the steps:
(a) adjusting the pH of a composition comprising L-NDDP, so that the pH is made acidic, said platinum complex having the formula

DACH-Pt-X₂

where DACH is 1,2-diaminocyclohexane and each X is independently-halogen or a lipid ligand; and
(b) after a predetermined time, adjusting the acidic pH of the composition of step (a) to a pH greater than 7.

In another embodiment, the liposomal platinum complex is formed by a method, said method comprising comprising the steps:
(a) adjusting the pH of a composition containing L-NDDP in the presence of sodium chloride, so that the pH is made acidic, said platinum complex having the formula

DACH-Pt-Cl₂

where DACH is 1,2-diaminocyclohexane; and
(b) after a predetermined time, adjusting the acidic pH of the composition of step (a) to a pH greater than 7.

Lipids useful in the present invention as liposomal lipid components of the liposomal platinum complexes include, but are not limited to, phospholipids, glycolipids, glycosphingolipids and sterols. Representative examples of glycolipids useful as liposomal lipid components include, but are not limited to, glycosphingolipids, such as ceramides, cerebrosides and gangliosides. Representative examples of sterols useful as liposomal lipid components include, but are not limited to, cholesterol.

In one embodiment, the liposomal platinum complexes of the present invention comprise two or more different liposomal lipid components.

In a specific embodiment, the liposomal platinum complexes of the present invention comprise two different liposomal lipid components.

In a preferred embodiment, the liposomal lipid component is a phospholipid. Phospholipids useful in the invention as liposomal lipid components include, but are not limited to, phosphatidyl cholines, phosphatidyl glycerols, phosphatidyl ethanolamines and sphingolipids, particularly sphingomyelin.

Representative examples of phospholipids useful as liposomal lipid components of the invention include, but are not limited to, dimyristoyl phosphatidyl choline (DMPC), egg phosphatidyl choline, dilauryloyl phosphatidyl choline, dipalmitoyl phosphatidyl choline, distearoyl phosphatidyl choline, 1-myristoyl-2-palmitoyl phosphatidyl choline, 1-palmitoyl-2-myristoyl phosphatidyl choline, 1-palmitoyl-2-stearoyl phosphafidyl choline, 1-stearoyl-2-palmitoyl phosphatidyl choline, dioleoyl phosphatidyl choline, dimyristoyl phosphatidyl glycerol (DMPG), dilauryloyl phosphatidyl glycerol, dioleyl phosphatidyl glycerol, dipalmitoyl phosphatidyl glycerol, distearoyl phosphatidyl glycerol, 1-myristoyl-2-palmitoyl phosphatidyl glycerol, 1-palmitoyl-2-myristoyl phosphatidyl glycerol, 1-palmitoyl-2-stearoyl phosphatidyl glycerol, 1-stearoyl-2-palmitoyl phosphatidyl glycerol, dioleoyl phosphatidyl glycerol, dimyristoyl phosphatidyl ethanolamine, dipalmitoyl phosphatidyl ethanolamine, brain sphingomyelin, dipalmitoyl sphingomyelin, and distearoyl sphingomyelin.

In one embodiment, the phospholipid is an acidic phospholipid.

In a preferred embodiment, the acidic phospholipid is DMPG.

Preferred phospholipids which are useful as liposomal lipid components of the invention, include, but are not limited to, phosphatidylglycerols and phosphatidylcholines. The most preferred phosphatidylglycerol is one consisting essentially of DMPG and the most preferred phosphatidylcholine is one consisting essentially of DMPC. In a preferred embodiment, the liposomal lipid compositions of the present invention have liposomes comprising a mixture of DMPG and DMPC as liposomal lipid components, preferably in a molar ratio between 1 to 10 and 10 to 1, more preferably DMPG and DMPC in a molar ratio of 3 to 7, respectively.

The liposomal platinum complexes of the present invention may contain the platinum complex and the liposomal lipid component in a molar ratio (of platinum complex to lipid component) between 1 to 2 and 1 to 30, preferably between 1 to 5 and 1 to 20, most preferably between 1 to 10 and 1 to 15.

The liposomes of the liposomal platinum complexes can be multilamellar, unilamellar or have an undefined lamellar construction. A pharmaceutical composition comprising an amount of a liposomal platinum complex effective to treat cancer, and a pharmaceutically acceptable carrier or vehicle can be administered for the treatment of cancer.

The liposomal platinum complexes of the invention may further comprise capecitabine entrapped within the liposome of the liposomal platinum complex.

The liposomal platinum complexes of the invention can further comprise a surfactant, said surfactant being nonionic, anionic, or cationic. Such liposomes can have median diameters of less than 1 μm. Examples of surfactants useful in the invention include, but are not limited to, sorbitan polyoxyethylene carboxylates, such as sorbitan polyoxyethylene monooleate and sorbitan polyoxyethylene monolaurate; sorbitan esters of common fatty acids, such as sorbitan monooleate, sorbitan monopalmitate and sorbitan monolaurate; polyoxyethylene ethers, such as polyoxyethylene monolauryl ether, polyoxyethylene monopalmityl ether, polyoxyethylene monostearyl ether and polyoxyethylene monooleyl ether; and block copolymers, such as those comprising ethylene oxide and propylene oxide.

Liposomal platinum complexes of the invention having a submicron diameter can be prepared by adding a surfactant to a solution of the liposomal lipid component(s) and a platinum complex. The surfactant can be present in an amount between 0.1 mole % to 5 mole % of the total amount of the liposomal lipid component(s). In one embodiment, the surfactant is present in an amount between 0.5 mole % and 4 mole % of the total amount of the liposomal lipid component(s). In a preferred embodiment, the surfactant is present in an amount between 1.5 mole % and 3 mole % of the total amount of the liposomal lipid component(s).

The preparation of submicron diameter liposomes comprising an anticancer agent, a surfactant and a phospholipid is described in U.S. Pat. No. 5,902,604, which is incorporated by reference herein in its entirety. A general procedure useful for the preparation of L-NDDP comprising liposomes of submicron diameter is presented in the examples section below.

In one embodiment, the surfactant is a nonionic surfactant.

In another embodiment, the nonionic surfactant is a polyoxyethylene sorbitan carboxylate.

In a specific embodiment, the nonionic surfactant is polyoxyethylene sorbitan monooleate.

In another specific embodiment, the nonionic surfactant is polyoxyethylene sorbitan monolaurate.

The submicron diameter liposomal platinum complexes of the invention can possess valuable pharmacological properties. Submicron liposomal formulations do not occlude capillaries of the circulatory system of a subject and are therefore particularly useful in parenteral and, more particularly, intravenous modes of administration.

Thus, submicron diameter liposomal platinum complexes of are especially useful when administered in the combination therapies of the present invention for treating cancer.

In a specific embodiment, a liposomal platinum complex may further comprise capecitabine or a pharmaceutically acceptable salt thereof, such that both a platinum complex, and capecitabine or a pharmaceutically acceptable salt thereof, are both entrapped in the same liposome. Such liposomal compositions may be prepared using the methodology disclosed in Section 6.1 herein under the heading “Preparation of L-NDDP,” by adding capecitabine or a pharmaceutically acceptable salt thereof, to the chloroform solution of Method I or to the tert-butanol solution of Method II and carrying out the method as indicated.

### 5.2 Capecitabine

Capecitabine is a fluoropyridine carbamate analog which acts as a prodrug for 5-fluorouracil. Capecitabine is relatively non-cytotoxic in vitro and is converted in vivo in both tumor cells and normal cells to 5-fluorouracil via a cascade of enzymatic reactions. It is believed that 5-fluorouracil blocks the methylation reaction which converts deoxytridylic acid to thymidylic acid. In this manner, the administration of capecitabine results in the interference of the synthesis of DNA, and to a lesser extent, the formation of RNA. Since DNA and RNA are essential for cell division and growth, the cytotoxic effect of capecitabine may due to the creation of a thymine deficiency which provokes unbalanced growth and the death of the cell. The effect of DNA and RNA deprivation are most marked in more rapidly dividing cells.

Capecitabine can be obtained commercially (Roche, Nutley, N.J.) or may be prepared using various methods known to one skilled in the art of synthetic organic chemistry. Such methods include, but are not limted to, those disclosed in U.S. Pat. No. 4,966,891 to Fujiu et al. and U.S. Pat. No. 5,472,949 to Arasaki et al.

As used in the present invention, capecitabine can be formulated as a pharmaceutically acceptable salt. The phrase “pharmaceutically acceptable salt,” as used herein, refers to a pharmaceutically acceptable organic or inorganic acid or base salt of an organic chemical compound. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. The counterion may be any organic or inorganic moiety that stabilizes the charge on the parent compound. Furthermore, a pharmaceutically acceptable salt may have more than one charged atom in its structure. In this instance the pharmaceutically acceptable salt can have multiple counterions. Hence, a pharmaceutically acceptable salt can have one or more charged atoms and/or one or more counterions.

### 5.3 Combination Chemotherapy

The combination therapies of the present invention comprise the administration of a liposomal platinum complex and capecitabine or a pharmaceutically acceptable salt thereof. In one embodiment, the combination therapies of the invention comprise the sequential administration of a liposomal platinum complex and capecitabine or a pharmaceutically acceptable salt thereof. In another embodiment, the combination therapies of the invention comprise the administration of a pharmaceutical composition comprising a pharmaceutically acceptable carrier, a liposomal platinum complex and capecitabine or a pharmaceutically acceptable salt thereof.

For ease of reference, the liposomal platinum complexes of the invention, capecitabine, a pharmaceutically acceptable salt of capecitabine, or any one or more of the foregoing will be referred to as the “combination anticancer agents of the invention.”

The liposomal platinum complex and capecitabine or a pharmaceutically acceptable salt thereof, can act additively or synergistically (i.e., the combination of a liposomal platinum complex and capecitabine is more effective than the additive effects of both of these agent when each is administered as monotherapy). A synergistic combination of L-NDDP and capecitabine permits the use of lower dosages of one or more of these agents and/or less frequent administration of said agents to a subject with cancer. The ability to utilize lower dosages of L-NDDP and/or capecitabine, and/or to administer said agents less frequently can reduce the toxicity associated with the administration of said agents to a subject without reducing the efficacy of said agents in the treatment of cancer. In addition, a synergistic effect can result in the improved efficacy of these agents in the treatment of cancer and/or the reduction of adverse or unwanted side effects associated with the use of either agent alone.

In one embodiment, the combination anticancer agents of the invention may act synergistically when administered in doses typically employed when such agents are used as monotherapy for the treatment of cancer. In another embodiment, the combination anticancer agents of the invention may act synergistically when administered in doses that are less than doses typically employed when such agents are used as monotherapy for the treatment of cancer.

### 5.4 Pharmaceutical Compositions and Therapeutic Administration

In other aspects, the present invention provides pharmaceutical compositions comprising the combination anticancer agents of the invention. The pharmaceutical compositions are suitable for veterinary or human administration.

In one embodiment, a composition of the invention comprises one of the combination anticancer agents of the invention and a pharmaceutically acceptable carrier or vehicle.

In a specific embodiment, a pharmaceutical composition of the invention comprises capecitabine or a pharmaceutically acceptable salt thereof and a pharmaceutically acceptable carrier or diluent. In another specific embodiment, a pharmaceutical composition of the invention comprises a liposomal platinum complex and a pharmaceutically acceptable carrier or diluent.

In one embodiment, a pharmaceutical composition of the invention comprises an amount of a liposomal platinum complex, and an amount of capecitabine or a pharmaceutically acceptable salt thereof, wherein said amounts are together effective to treat cancer.

In another embodiment, a pharmaceutical composition of the invention comprises a synergistic amount of the combination anticancer agents of the invention. In one embodiment, a synergistic combination may contain: (a) an amount of a liposomal platinum complex which is less than the amount of said liposomal platinum complex effective to treat cancer when said liposomal platinum complex is administered as a single-agent, and/or (b) an amount of capecitabine or a pharmaceutically acceptable salt thereof, which is less than the amount of capecitabine or a pharmaceutically acceptable salt thereof, effective to treat cancer when administered as a single-agent. In another embodiment, a synergistic combination may contain an amount of a liposomal platinum complex and/or an amount of capecitabine or a pharmaceutically acceptable salt thereof, which is similar to the amounts used when each of these agents are administered as monotherapy for the treatment of cancer.

The pharmaceutical compositions of the present invention comprise one or more of the combination anticancer agents of the invention, and can be in any form that allows for the composition to be administered to a subject. The subject of the combination therapy of the present invention is preferably an animal, including, but not limited to a human, mammal, or non-human animal, such as a cow, horse, sheep, pig, fowl, cat, dog, mouse, rat, rabbit, guinea pig, etc., and is more preferably a mammal, and most preferably a human.

The compositions of the invention can be in the form of a solid, liquid or gas (aerosol). Typical routes of administration may include, without limitation, oral, topical, parenteral, sublingual, rectal, vaginal, ocular, and intranasal. Parenteral administration includes subcutaneous injections, intravenous, intramuscular, intraperitoneal, intrapleural, intrasternal injection or infusion techniques. Preferably, the compositions are administered parenterally, most preferably intravenously. Pharmaceutical compositions of the invention can be formulated so as to allow the combination anticancer agents of the invention to be bioavailable upon administration of the composition to a subject. Compositions can take the form of one or more dosage units, where for example, a tablet can be a single dosage unit, and a container of the combination anticancer agents of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of cancer the subject is in need of treatment for, the use of the composition as part of a multi-drug regimen, the particular form of each of the combination anticancer agents of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

The composition may be intended for oral administration, and if so, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as ethyl cellulose, carboxymethylcellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the pharmaceutical composition is in the form of a capsule, e.g., a gelatin capsule, it can contain, in addition to materials of the above type, a liquid carrier such as polyethylene glycol, cyclodextrin or a fatty oil.

The pharmaceutical composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is a preferred adjuvant. An injectable composition is preferably sterile.

The amount of the combination anticancer agents of the invention effective in the treatment of a particular disorder or condition will depend on the nature of the disorder or condition, and can be determined by standard clinical techniques. In addition, in vitro or in vivo assays can optionally be employed to help identify optimal dosage ranges. The precise doses to be employed in the compositions will also depend on the route of administration, and the seriousness of the disease or disorder, and should be decided according to the judgment of the practitioner and each patient's circumstances.

In a preferred embodiment, the combination anticancer agents of the invention are administered in doses commonly employed when such agents are used as monotherapy for the treatment of cancer.

In another embodiment, the combination anticancer agents of the invention act synergistically and are administered in doses that are less than the doses commonly employed when such agents are used as monotherapy for the treatment of cancer.

In one embodiment, the pharmaceutical compositions comprise an amount of each the combination anticancer agents of the invention which together are effective to treat cancer. In another embodiment, the pharmaceutical compositions comprise an amount of the combination anticancer agents of the invention which are effective to treat cancer when each of the anticancer agents are administered separately as monotherapy. Typically, the compositions of the invention comprise at least about 0.01% of the combined combination anticancer agents of the invention by weight of the composition. When intended for oral administration, this amount can be varied to be between 0.1% and 80% by weight of the composition. Preferred oral compositions can comprise from between 4% and 50% of combined amount of the combination anticancer agents of the invention by weight of the composition. Preferred compositions of the present invention are prepared so that a parenteral dosage unit contains from between 0.01% and 2% by weight of the combined amount of the combination anticancer agents of the invention.

When used in the invention, a liposomal platinum complex can administered to a subject at dosages from about 1 mg/m² to about 1000 mg/m², from about 100 mg/m² to about 700 mg/m², preferably from about 200 mg/m² to about 500 mg/m². In one embodiment, the liposomal platinum complex is administered at doses from about 7.5 mg/m² to about 390 mg/m² once every three weeks, or alternatively at doses from about 300 mg/m² to about 500 mg/m² once every four weeks, depending on various parameters, including, but not limited to, the cancer being treated, the patient's general health, and the administering physician's discretion. In specific embodiments, the dosages of the liposomal platinum complex administered to a subject are about 25 mg/m², about 50 mg/m², about 75 mg/m², about 100 mg/m², about 125 mg/m², about 150 mg/m², about 175 mg/m², about 200 mg/m², about 225 Mg/m², about 250 mg/m², about 275 mg/m², about 300 mg/m², about 325 mg/m², about 350 mg/m², about 375 mg/m², about 400 mg/m², about 425 mg/m², about 450 mg/m², about 475 mg/m², about 500 mg/m², about 525 mg/m², about 550 mg/m², about 575 mg/m², about 600 mg/m², about 625 mg/m², about 650 mg/m², about 675 mg/m², about 700 mg/m², about 725 mg/m², about 750 mg/m², about 775 mg/m², about 800 mg/m², about 825 mg/m², about 850 mg/m², about 875 mg/m², about 900 mg/m², about 925 mg/m², about 950 mg/m², about 975 mg/m², or about 1000 mg/m².

When used in the invention, capecitabine or a pharmaceutically acceptable salt thereof, can be administered to a subject at dosages from about 100 mg/m² to about 3000 mg/m². In a preferred embodiment, capecitabine is administered at a dosage of about 1250 mg/m² administered orally twice daily (equivalent to a 2500 mg/m² daily dosage) for two weeks followed by a one week rest period, given as three-week cycles. In another embodiment, capecitabine is administered at a dose of about 200 mg/m² over about three minutes. In specific embodiments, the dosages of capecitabine administered to a subject are about 100 mg/m², about 200 mg/m², about 300 mg/m², about 400 mg/m², about 500 mg/m², about 600 mg/m², about 700 mg/m², about 800 mg/m², about 900 mg/m², about 1000 mg/m², about 1100 mg/m², about 1200 mg/m², about 1300 mg/m², about 1400 mg/m², about 1500 mg/m², about 1600 mg/m², about 1700 mg/m², about 1800 mg/m², about 1900 mg/m², about 2000 mg/m², 2100 mg/m², about 2200 mg/m², about 2300 mg/m², about 2400 mg/m², about 2500 mg/m², about 2600 mg/m², about 2700 mg/m², about 2800 mg/m², about 2900 mg/m², or about 3000 mg/m².

The combination anticancer agents of the invention can be administered by any convenient route, for example by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering the combination anticancer agents of the invention. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. The preferred mode of administration is left to the discretion of the practitioner, and will depend in-part upon the site of the medical condition (such as the site of cancer, a cancerous tumor or a pre-cancerous condition).

In one embodiment, the liposomal platinum complex is administered intravenously, intrapleurally, intra-arterially or intraperitoneally. In a preferred embodiment, the liposomal platinum complex is administered intravenously.

In a preferred embodiment, capecitabine is administered orally.

In a specific embodiment, the invention provides a combination therapy regimen useful for treating cancer, each cycle of said regimen comprising: (a) administering to a subject in need thereof on day 1: L-NDDP intravenously at a dose of about 50 to about 500 mg/m², followed by capecitabine orally at a dose of about 1000 mg/m² twice daily (for a total daily dose of 2000 mg/m²) and (b) administering to said subject every day on days 2-14: capecitabine orally at a dose of about 1000 mg/m² twice daily (for a total daily dose of 2000 mg/m²). In one embodiment, a subject can receive from 1 to about 12 cycles.

In specific embodiments, it can be desirable to administer the combination anticancer agents of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as sialastic membranes, or fibers. In one embodiment, administration can be by direct injection at the site (or former site) of a cancer, tumor, or precancerous tissue. In certain embodiments, it can be desirable to introduce the combination anticancer agents of the invention into the central nervous system by any suitable route, including intraventricular and intrathecal injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the combination anticancer agents of the invention can be formulated in suppository form, with traditional binders and carriers such as triglycerides.

In yet another embodiment, the combination anticancer agents of the invention can be delivered in a controlled release system. In one embodiment, a pump can be used (see Langer, supra; Sefton, CRC Crit. Ref. Biomed. Eng. 1987, 14:201; Buchwald et al., Surgery 88:507 (1980); Saudek et al., N. Engl. J. Med. 1989, 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 1983, 23:61; see also Levy et al., Science 1985, 228:190; During et al., Ann. Neurol. 1989, 25:351; Howard et al., J. Neurosurg. 1989, 71:105). In yet another embodiment, a controlled-release system can be placed in proximity of the target of the combination anticancer agents of the invention, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 1990, 249:1527-1533) can be used.

The term “carrier” refers to a diluent, adjuvant or excipient, with which one or more of the combination anticancer agents of the invention can be administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the combination anticancer agents of the invention and pharmaceutically acceptable carriers are sterile. Water is a preferred carrier when the anticancer compounds of the invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in E. W. Martin “Remington's Pharmaceutical Sciences” Mack Publishing Co., 18^{th} Edition (1990).

Sustained or directed release compositions that can be formulated include, but are not limited to, the liposomal platinum complexes of the invention, liposomally encapsulated capecitabine, and other formulations where capecitabine or a pharmaceutically acceptable salt thereof is protected with differentially degradable coatings, e.g., by microencapsulation, multiple coatings, etc. It is also possible to freeze-dry the compositions and use the lyophilizates obtained, for example, for the preparation of products for injection.

In a preferred embodiment, the combination anticancer agents of the invention are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the combination anticancer agents of the invention are to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving complex are also suitable for orally administered compositions of the invention. In these later platforms, fluid from the environment surrounding the capsule is imbibed by the driving complex, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers are preferably of pharmaceutical grade.

The pharmaceutical compositions of the invention can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a total concentration of the combination anticancer agents of the invention of from between 0.01% and 10% w/v (weight per unit volume of composition).

The compositions can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions may consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of the compositions can be delivered in single phase, bi-phasic, or tri-phasic systems in order to deliver the composition. Delivery of the aerosol includes the necessary container, activators, valves, subcontainers, Spacers and the like, which together can form a kit. Preferred aerosols can be determined by one skilled in the art, without undue experimentation.

Whether in solid, liquid or gaseous form, the compositions of the present invention can comprise an additional therapeutically active agent selected from among those including, but not limited to, an antiemetic agent, a hematopoietic colony stimulating factor, an anti-depressant and an analgesic agent.

The pharmaceutical compositions can be prepared using methodology well known in the pharmaceutical art. For example, a composition intended to be administered by injection can be prepared by combining the combination anticancer agents of the invention with water so as to form a solution. A surfactant can be added to facilitate the formation of a homogeneous solution or suspension. Surfactants are complexes that can non-covalently interact with the combination anticancer agents of the invention so as to facilitate dissolution or homogeneous suspension of the combination anticancer agents of the invention in the aqueous delivery system.

In one embodiment, the pharmaceutical compositions of the present invention may comprise one or more known therapeutically active agents.

In one embodiment, the pharmaceutical compositions of the present invention can be administered prior to, at the same time as, or after an antiemetic agent, or on the same day, or within 1 hour, 2 hours, 12 hours, 24 hours, 48 hours or 72 hours of each other.

In another embodiment, the pharmaceutical compositions of the present invention can be administered prior to, at the same time as, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In another embodiment, the pharmaceutical compositions of the present invention can be administered prior to, at the same time as, or after an opioid or non-opioid analgesic agent, or on the same day, or within 1 hour, 2 hours, 12 hours, 24 hours, 48 hours or 72 hours of each other.

In another embodiment, the pharmaceutical compositions of the present invention can be administered prior to, at the same time as, or after an anti-depressant agent, or on the same day, or within 1 hour, 2 hours, 12 hours, 24 hours, 48 hours or 72 hours of each other.

The combination anticancer agents of the present invention can be administered concurrently or sequentially to a subject. The anticancer agents of the present invention can also be cyclically administered. Cycling therapy involves the administration of one anticancer agent of the invention for a period of time, followed by the administration of a second anticancer agent of the invention for a period of time and repeating this sequential administration, i.e., the cycle, in order to reduce the development of resistance to one or both of the combination anticancer agents of the invention, to avoid or reduce the side effects of one or both of the combination anticancer agents of the invention, and/or to improve the efficacy of the treatment.

In a preferred embodiment, the combination anticancer agents of the invention are administered concurrently to a subject in separate compositions. The combination anticancer agents of the invention may be administered to a subject by the same or different routes of administration.

When the combination anticancer agents of the invention are administered to a subject concurrently, the term “concurrently” is not limited to the administration of the combination anticancer agents of the invention at exactly the same time, but rather it is meant that they are administered to a subject in a sequence and within a time interval such that they can act synergistically to provide an increased benefit than if they were administered otherwise. For example, the combination anticancer agents of the invention may be administered at the same time or sequentially in any order at different points in time; however, if not administered at the same time, they should be administered sufficiently close in time so as to provide the desired therapeutic effect, preferably in a synergistic fashion. The combination anticancer agents of the invention can be administered separately, in any appropriate form and by any suitable route. When the components of the combination therapies of the are not administered in the same pharmaceutical composition, it is understood that they can be administered in any order to a subject in need thereof. For example, a liposomal platinum complex can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of capecitabine, to a subject in need thereof. In various embodiments the combination anticancer agents of the invention are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the combination anticancer agents of the invention are administered within the same office visit. In another embodiment, the combination anticancer agents of the invention are administered 1 minute to 24 hours apart.

In one embodiment, the combination anticancer agents of the invention may be administered along with one or more known therapeutically active agents.

### 5.5 Kits

The invention encompasses kits that can simplify the administration of the combination anticancer agents of the invention or composition of the invention to a subject.

A typical kit of the invention comprises unit dosages of the combination anticancer agents of the invention. In one embodiment, the unit dosage form is in a container, which can be sterile, containing an effective amount of one of the combination anticancer agents of the invention and a pharmaceutically acceptable carrier or vehicle. In another embodiment, the unit dosage form is in a container containing an effective amount of one of the anticancer agent of the invention as a lyophilate. In this instance, the kit can further comprise another container which contains a solution useful for the reconstitution of the lyophilate. In one embodiment, ths kit comprises an acidic solution useful for the reconstitution of L-NDDP, preferably an acidic saline solution. The kit can also comprise a basic solution useful for stopping the acid-catalyzed degradation of L-NDDP, such as a buffer solution, more preferably phosphate buffered saline. The kit can also comprise a label or printed instructions for use of the combination anticancer agents of the invention. In one embodiment, the kit comprises two containers: (a) a first container containing an unit dosage form of a liposomal platinum complex, and (b) a second container containing a unit dosage form of capecitabine or a pharmaceutically acceptable salt thereof. In another embodiment the kit comprises a container containing a therapeutically active agent such as an antiemetic agent, a hematopoietic colony-stimulating factor, an analgesic agent or an anxiolytic agent.

In a further embodiment, the kit comprises a unit dosage form of a pharmaceutical composition of the invention.

Kits of the invention can further comprise one or more devices that are useful for administering the unit dosage forms of the combination anticancer agents of the invention or a pharmaceutical composition of the invention. Examples of such devices include, but are not limited to, a syringe, a drip bag, a patch or an enema, which optionally contain the unit dosage forms.

### 5.6 Therapeutic Uses

The present invention provides methods for treating cancer, said methods comprising administering to a subject in need thereof a liposomal platinum complex (e.g., L-NDDP) and capecitabine or a pharmaceutically acceptable salt thereof.

In one embodiment, the present invention provides a method for treating cancer, said method comprising sequentially administering to a subject in need thereof an amount of a liposomal platinum complex, and an amount of capecitabine, wherein said amounts are together effective to treat cancer.

In a further embodiment, the invention provides a method for treating cancer said method comprising administering to a subject in need thereof a liposomal platinum complex and capecitabine or a pharmaceutically acceptable salt thereof, wherein the liposomal platinum complex and the capecitabine act synergistically.

In a specific embodiment, the present invention provides a method for treating cancer, said method comprising administering to a subject in need thereof, an amount of a pharmaceutical composition comprising the combination anticancer agents of the invention, said amount effective to treat cancer.

### 5.6.1 Treatment of Cancer

Cancer can be treated or prevented by administration of amounts of the combination anticancer agents of the invention that are together effective to treat cancer or by administration of an amount of a pharmaceutical composition comprising amounts of the combination anticancer agents of the invention that are together effective to treat cancer.

### 5.6.1.1 Therapeutic Methods

In a preferred embodiment, the present invention provides methods for treating cancer, including but not limited to: killing a cancer cell or neoplastic cell; inhibiting the growth of a cancer cell or neoplastic cell; inhibiting the replication of a cancer cell or neoplastic cell; or ameliorating a symptom thereof, said methods comprising administering to a subject in need thereof an amount of the combination anticancer agents of the invention effective to treat cancer.

In one embodiment, the invention provides a method for treating cancer, said method comprising administering to a subject in need thereof an amount of a pharmaceutical composition, said composition comprising a pharmaceutically acceptable carrier or diluent, a amount of a liposomal platinum complex, and an amount of capecitabine or a pharmaceutically acceptable salt thereof, wherein said amounts are together effective to treat cancer.

In another embodiment, the invention provides a method for treating cancer, said method comprising (a) administering to a subject in need thereof an amount of a first pharmaceutical composition comprising a liposomal platinum complex and a pharmaceutically acceptable carrier or diluent; and (b) administering to said subject an amount of a second pharmaceutical composition comprising capecitabine or a pharmaceutically acceptable salt thereof, and a pharmaceutically acceptable carrier or diluent, wherein said amounts are together effective to treat cancer.

The combination anticancer agents of the invention can be used accordingly in a variety of settings for the treatment of various cancers.

In a specific embodiment, the subject in need of treatment has previously undergone treatment for cancer. Such previous treatments include, but are not limited to, prior chemotherapy, radiation therapy, surgery or immunotherapy, such as cancer vaccines.

In another embodiment, the cancer being treated is a cancer which has demonstrated sensitivity to platinum therapy or is known to be responsive to platinum therapy. Such cancers include, but are not limited to, small-cell lung cancer, non-small cell lung cancer, ovarian cancer, breast cancer, bladder cancer, testicular cancer, head and neck cancer, colorectal cancer, Hodgkin's disease, leukemia, osteogenic sarcoma, and melanoma.

In still another embodiment, the cancer being treated is a cancer which has demonstrated resistance to platinum therapy or is known to be refractory to platinum therapy. Such refractory cancers can include, but are not limited to, cancers of the cervix, prostate, and esophagus. A cancer may be determined to be refractory to a therapy when at least some significant portion of the cancer cells are not killed or their cell division are not arrested in response to therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased. Such cancers can include, but are not limited to, cancers of the cervix, prostate, and esophagus.

Other cancers that can be treated with the combination anticancer agents of the invention include, but are not limited to, cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epithelial carcinoma
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
melanoma
neuroblastoma
retinoblastoma
blood-borne cancers, including but not limited to:
acute lymphoblastic leukemia (“ALL”)
acute lymphoblastic B-cell leukemia
acute lymphoblastic T-cell leukemia
acute myeloblastic leukemia (“AML”)
acute promyelocytic leukemia (“APL”)
acute monoblastic leukemia
acute erythroleukemic leukemia
acute megakaryoblastic leukemia
acute myelomonocytic leukemia
acute nonlymphocyctic leukemia
acute undifferentiated leukemia
chronic myelocytic leukemia (“CML”)
chronic lymphocytic leukemia (“CLL”)
hairy cell leukemia
multiple myeloma
acute and chronic leukemias:
lymphoblastic
myelogenous
lymphocytic
myelocytic leukemias
Lymphomas:
Hodgkin's disease
non-Hodgkin's Lymphoma
Multiple mycloma
Waldenström's macroglobulinemia
Heavy chain disease
Polycythemia vera

In one embodiment, the cancer is selected from the group consisting of pancreatic cancer, colorectal cancer, mesothelioma, a malignant pleural effusion, peritoneal carcinomatosis, peritoneal sarcomatosis, renal cell carcinoma, small cell lung cancer, non-small cell lung cancer, testicular cancer, bladder cancer, breast cancer, head and neck cancer, and ovarian cancer.

In a preferred embodiment the cancer is pancreatic cancer or colorectal cancer.

### 5.6.1.2 Prophylactic Methods

The combination anticancer agents of the invention can also be administered to prevent progression to a neoplastic or malignant state, including but not limited to the cancers listed in Table 1. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79). Hyperplasia is a form of controlled cell proliferation involving an increase in cell number in a tissue or organ, without significant alteration in structure or function. For example, endometrial hyperplasia often precedes endometrial cancer and precancerous colon polyps often transform into cancerous lesions. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

The prophylactic use of the combination anticancer agents of the invention are also indicated in some viral infections that may lead to cancer. For example, human papilloma virus can lead to cervical cancer (see, e.g., Hemandez-Avila et al., Archives of Medical Research (1997) 28:265-271), Epstein-Barr virus (EBV) can lead to lymphoma (see, e.g., Herrmann et al., J Pathol (2003) 199(2): 140-5), hepatitis B or C virus can lead to liver carcinoma (see, e.g., El-Serag, J Clin Gastroenterol (2002) 35(5 Suppl 2):S72-8), human T cell leukemia virus (HTLV)-I can lead to T-cell leukemia (see e.g., Mortreux et al., Leukemia (2003) 17(l):26-38), human herpesvirus-8 infection can lead to Kaposi's sarcoma (see, e.g., Kadow et al., Curr Opin Investig Drugs (2002) 3(11):1574-9), and Human Immune deficiency Virus (HIV) infection contribute to cancer development as a consequence of immunodeficiency (see, e.g., Dal Maso et al., Lancet Oncol (2003) 4(2): 110-9).

In other embodiments, a patient which exhibits one or more of the following predisposing factors for malignancy can treated by administration of an amount of the combination anticancer agents of the invention which are together effective to treat cancer: a chromosomal translocation associated with a malignancy (e.g., the Philadelphia chromosome for chronic myelogenous leukemia, t(14;18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (e.g., smoking, and inhalation of or contacting with certain chemicals).

In another specific embodiment, the combination anticancer agents of the invention are administered to a human patient to prevent progression to breast, colon, ovarian, or cervical cancer.

### 5.6.2 Multi-Modality Therapy for Cancer

The combination anticancer agents of the invention can be administered to a subject that has undergone or is currently undergoing one or more additional anticancer treatment modalities including, but not limited to, surgery, radiation therapy, or immunotherapy, such as cancer vaccines.

In one embodiment, the invention provides methods for treating cancer comprising (a) administering to a subject in need thereof an amount of a combination therapy of the invention effective to treat cancer; and (b) administering to said subject one or more additional anticancer treatment modalities including, but not limited to, surgery, radiation therapy, or immunotherapy, such as a cancer vaccine.

In one embodiment, the additional anticancer treatment modality is radiation therapy.

In another embodiment, the additional anticancer treatment modality is surgery.

In still another embodiment, the additional anticancer treatment modality is immunotherapy.

In a specific embodiment, the combination anticancer agents of the invention are administered concurrently with radiation therapy. In another specific embodiment, the additional anticancer treatment modality is administered prior or subsequent to the combination anticancer agents of the invention, preferably at least an hour, five hours, 12 hours, a day, a week, a month, more preferably several months (e.g., up to three months), prior or subsequent to administration of the combination anticancer agents of the invention.

When the additional anticancer treatment modality is radiation therapy, any radiation therapy protocol can be used depending upon the type of cancer to be treated. For example, but not by way of limitation, X-ray radiation can be administered; in particular, high-energy megavoltage (radiation of greater that 1 MeV energy) can be used for deep tumors, and electron beam and orthovoltage X-ray radiation can be used for skin cancers. Gamma-ray emitting radioisotopes, such as radioactive isotopes of radium, cobalt and other elements, can also be administered.

Additionally, the invention provides methods of treatment of cancer using the combination anticancer agents of the invention as an alternative to chemotherapy or radiation therapy where the chemotherapy or the radiation therapy has proven or can prove too toxic, e.g., results in unacceptable or unbearable side effects, for the subject being treated. The subject being treated can, optionally, be treated with another anticancer treatment modality such as surgery, radiation therapy or immunotherapy, depending on which treatment is found to be acceptable or bearable.

The combination anticancer agents of the invention can also be used in an in vitro or ex vivo fashion, such as for the treatment of certain cancers, including, but not limited to leukemias and lymphomas, such treatment involving autologous stem cell transplants. This can involve a multi-step process in which the animal's autologous hematopoietic stem cells are harvested and purged of all cancer cells, the patient's remaining bone-marrow cell population is then eradicated via the administration of high doses of the combination anticancer agents of the invention and/or high dose radiation therapy, and the stem cell graft is infused back into the animal. Supportive care is then provided while bone marrow function is restored and the subject recovers.

### 5.7 Other Therapeutic Agents

The present methods can further comprise the administration of the combination anticancer agents of the invention and another therapeutically active agent or a pharmaceutically acceptable salt thereof. In a preferred embodiment, the combination anticancer agents of the invention are administered concurrently with the administration of one or more other therapeutically active agents, which can be part of the same composition or in a different composition from that of the combination anticancer agents of the invention (which can be in the same or different pharmaceutical compositions). In another embodiment, the combination anticancer agents of the invention are administered prior to, concurrent with, or subsequent to the administration of one or more other therapeutically active agents. Kits comprising the combination anticancer agents of the invention, preferably purified, and one or more other therapeutically active agents, in one or more containers are also provided.

In the present methods for treating cancer the other therapeutically active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

In a preferred embodiment, the antiemetic agent is granisetron or ondansetron.

In another embodiment, the other therapeutically active agent can be an hematopoietic colony stimulating factor. Suitable hematopoietic colony stimulating factors include, but are not limited to, filgrastim, sargramostim, molgramostim and epoietin alfa.

In still another embodiment, the other therapeutically active agent can be an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the other therapeutically active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

### 6. EXAMPLES

### 6.1 Example 1

### Method for the Preparation of Liposomal Platinum Complexes

### Preparation of cis-bis-dichloro-DACH-Pt (II)

To a solution of K₂PtCl₄ in water (about 0.07 g/ml) is added 1,2-diaminocyclohexane (about 0.3 g/g K₂PtCl₄), the resulting reaction is stirred for about 8 hours at about 25° C., and the resulting yellow solid is removed by filtration. The solid is then washed sequentially with water, methanol and acetone, and dried in vacuo to provide cis-bis-dichloro-DACH-Pt (II).

### Preparation of sulfato-DACH-Pt H₂O

cis-bis-dichloro-DACH-Pt (II) is suspended in water (about 0.05 g/ml) and to the suspension is added a solution of Ag₂SO₄ in water (about 0.005 g/ml) and the resulting reaction is stirred in the dark for about 24 hours, then filtered. The filtrate is concentrated in vacuo and the resulting solid yellow residue is dried over P₂O₅ to provide sulfato-DACH-Pt H₂O.

### Preparation of cis-bis-neodecanoato-DACH-Pt (II)(NDDP)

To a solution of sulfato-DACH-Pt H₂O in water (about 0.04 g/ml) is added the potassium salt of neodecanoic acid (approximately 1 g per g of sulfato-DACH-Pt H₂O) and the resulting reaction is stirred for about 30 minutes at about 25° C., after which time a gummy mass is present. The reaction mixture is diluted with chloroform in an amount sufficient to dissolve the gummy mass and the resulting solution is transferred to a separatory funnel. The organic layer is collected, dried over MgSO₄, filtered and concentrated in vacuo to afford an off-white residue which is first dried in vacuo and then dried over P₂O₅ to provide NDDP as an off-white solid.

### Preparation of L-NDDP

Method I

NDDP and the liposomal lipid component(s) are combined in the desired ratios and taken up in chloroform. The resulting solution is concentrated in vacuo to afford a dried film which is then dispersed with an aqueous sodium chloride solution using methods including, but not limited to vigorous handshaking or vortexing, to provide a suspension which is subsequently centrifuged at about 30,000×g for about 45 minutes. The supernatant is discarded and the resulting solid is reconsituted in an appropriate reconstitution media to provide L-NDDP.

Method II

NDDP and the liposomal lipid component(s) are combined in the desired ratios and taken up in tert-butanol. The resulting solution is freeze-dried to provide a lyophilate which is subsequently reconstituted using an appropriate reconstitution media to provide L-NDDP.

### 6.2 Example 2

### Method for the Preparation of Liposomal Platinum Complexes having Submicron Diameter Liposomes

1) Prepare a first solution of NDDP in DMSO (approximately 100 mg/ml).

2) Prepare a second solution compring the liposomal lipid component(s) in a mixture of tert-butanol:water (9:1), the total lipid concentration being approximately 80 mg/ml.

3) Prepare a third solution by combining the first and second solutions in the necessary proportions to achieve the desired ratio of NDDP to liposomal lipid component(s).

4) Add the desired amount of surfactant to the third solution and filter the resulting fourth solution through a 0.22 μm pore filter of regenerated cellulose for sterilization (said filter can be purchased for example, from Micro Filtration Systems, Dublin, Calif.)

5) Freeze the filtered fourth solution in a bath consisting of dry ice/acetone and lyophilize for 48 hours to remove all DMSO and tert-butanol to provide a lyophilate.

6) Reconstitute the lyophilate of step by adding to the lyophilate a 37° C. saline solution, using approximately 1 ml of saline solution per mg NDDP.

### 7. REFERENCES CITED

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All references cited herein are incorporated by reference in their entirety and for all purposes to the same extent as if each individual publication or patent or patent application was specifically and individually indicated to be incorporated by reference in its entirety for all purposes.

1. A method for treating cancer, said method comprising:
(a) administering to a subject in need thereof an amount of L-NDDP; and
(b) administering to said subject an amount of capecitabine or a pharmaceutically acceptable salt thereof.

2. The method of claim 1 where the capecitabine or a pharmaceutically acceptable salt thereof, is administered at a time prior to the administration of L-NDDP.

3. The method of claim 1 where the capecitabine or a pharmaceutically acceptable salt thereof, is administered concurrently with L-NDDP.

4. The method of claim 1 where the capecitabine or a pharmaceutically acceptable salt thereof, is administered at a time subsequent to the administration of L-NDDP.

5. A method for treating cancer, said method comprising:
(a) administering to a subject in need thereof capecitabine or a pharmaceutically acceptable salt thereof; and
(b) administering to said subject a platinum complex having the formula
DACH-Pt-X₂
wherein said platinum complex is entrapped in a liposome, and where DACH is diaminocyclohexane and X is -halogen.

6. A method for treating cancer, said method comprising:
(a) administering to a subject in need thereof capecitabine or a pharmaceutically acceptable salt thereof; and
(b) administering to said subject a platinum complex having the formula
DACH-Pt-Cl₂
wherein said platinum complex is entrapped in a liposome, and where DACH is diaminocyclohexane.

7. A method for treating cancer, said method comprising
(a) administering to a subject in need thereof capecitabine or a pharmaceutically acceptable salt thereof; and
(b) administering to said subject a liposomal platinum complex, said liposomal platinum complex formed by a second method, said second method comprising making the pH of a composition comprising L-NDDP be acidic, and wherein said liposomal platinum complex comprises a platinum complex having the formula:
DACH-Pt-X₂
where DACH is 1,2-diaminocyclohexane and X is -halogen.

8. (canceled)

9. The method of claim 7, wherein the liposomal platinum complex of step (b) comprises a platinum complex having the formula:
DACH-Pt-Cl₂
where DACH is 1,2-diaminocyclohexane.

10. The method of claim 7 wherein said making comprises exposing the L-NDDP to a solution having an acidic pH.

11. The method of claim 7 wherein said second method further comprises before said making step, the step of entrapping NDDP in a liposome.

12. The method of claim 7 wherein said making of step (b) comprises reconstituting a lyophilized composition comprising NDDP and a liposomal lipid component, wherein said lyophilized composition did not contain liposomes at the time of lyophilization, and wherein said reconstitution is carried out in an acidic solution.

13. The method of claim 7 wherein said acidic pH of step (b) is between 2 and 6.5.

14. The method of claim 7 wherein said making comprises adding an acidic solution.

15. The method of claim 12 wherein said acidic solution comprises sodium chloride.

16. The method of claim 15 wherein said acidic solution is an aqueous solution.

17. A method for treating cancer, said method comprising:
(a) administering to a subject in need thereof capecitabine or a pharmaceutically acceptable thereof; and
(b) administering to said subject a liposomal platinum complex, said liposomal platinum complex formed by a second method, said second method comprising the steps:
(i) making the pH of a composition comprising L-NDDP be acidic; and
(ii) after a predetermined time, adjusting the acidic pH of the composition of step (i) to a pH greater than 7;
wherein said liposomal platinum complex comprises a platinum complex having the formula
DACH-Pt-X₂
where DACH is 1,2-diaminocyclohexane and X is -halogen.

18. (canceled)

19. The method of claim 17 where the liposomal platinum complex of step (b) comprises a platinum complex having the formula
DACH-Pt-Cl₂
where DACH is 1,2-diaminocyclohexane.

20. The method of claim 17 where the making of step (i) comprises adding an acidic solution.

21. The method of claim 20 wherein said acidic solution comprises sodium chloride.

22. The method of claim 21 wherein said acidic solution is an aqueous solution.

23. The method of claim 17 wherein said acidic pH of step (i) is between 2 and 6.5.

24. The method of claim 17 where the adjusting of step (ii) comprises adding a basic solution to the composition of step (i).

25. The method of claim 24 where the basic solution is a buffer solution.

26. The method of claim 25 where the buffer solution is phosphate buffered saline.

27. The method of claim 17 wherein said method further comprises before said making of step (i), the step of entrapping NDDP in a liposome.

28. The method of claim 11 wherein said entrapping is done in the presence of sodium chloride or chloroform.

29. The method of claim 17 wherein said making of step (i) comprises reconstituting a lyophilized composition comprising NDDP and a liposomal lipid component, wherein said lyophilized composition did not contain liposomes at the time of lyophilization, and wherein said reconstitution is carried out in an acidic solution.

30. A method for treating cancer, said method comprising:
(a) administering to a subject in need thereof an amount of a first pharmaceutical composition comprising L-NDDP and a pharmaceutically acceptable carrier or diluent; and
(b) administering to said subject an amount of a second pharmaceutical composition comprising capecitabine or a pharmaceutically acceptable salt thereof, and a pharmaceutically acceptable carrier or diluent.

31. The method of claim 30 where the first pharmaceutical composition is administered at a time prior to the administration of the second pharmaceutical composition.

32. The method of claim 30 where the first pharmaceutical composition is administered concurrently with the second pharmaceutical composition.

33. The method of claim 30 where the first pharmaceutical composition is administered at a time subsequent to the administration of the second pharmaceutical composition.

34. The method of claim 1 wherein the cancer is pancreatic cancer or colorectal cancer.

35. The method of claim 1 wherein the subject is a human.

36. The method of claim 1 wherein the time period elapsed between the administration of the L-NDDP and capecitabine or a pharmaceutically acceptable salt thereof is from 1 minute to 24 hours.

37. The method of claim 5, wherein the time period elapsed between the administration of said platinum complex and capecitabine a pharmaceutically acceptable salt thereof is from 1 minute to 24 hours.

38. The method of claim 7 wherein the time period elapsed between the administration of said liposomal platinum complex and capecitabine or a pharmaceutically acceptable salt thereof is from 1 minute to 24 hours.

39. The method of claim 30 wherein the time period elapsed between the administration of said first pharmaceutical composition and said second pharmaceutical composition is from 1 minute to 24 hours.

40. The method of claim 1 wherein the L-NDDP and/or capecitabine or a pharmaceutically acceptable salt thereof are in purified form.

41. The method of claim 5, wherein the platinum complex and/or capecitabine or a pharmaceutically acceptable salt thereof are in purified form.

42. The method of claim 7 wherein the liposomal platinum complex and/or capecitabine or a pharmaceutically acceptable salt thereof are in purified form.

43. A kit comprising: (a) a first container which contains a unit dosage form of capecitabine or a pharmaceutically acceptable salt thereof; and (b) a second container which contains a unit dosage form of L-NDDP.

44. The kit of claim 43 wherein the L-NDDP is in lyophilized form.

45. The kit of claim 44 further comprising a third container, the third container containing a solution useful for reconstitution of the L-NDDP.

46. The kit of claim 45 where the solution is an acidic solution.

47. The kit of claim 46 where the solution is an aqueous solution.

48. The kit of claim 47 where the aqueous solution comprises sodium chloride.

49. The kit of claim 45 further comprising a fourth container, the fourth container containing a basic solution useful for stopping acid-catalyzed degradation of L-NDDP.

50. The kit of claim 49 where the basic solution is a buffer solution.

51. The kit of claim 50 where the buffer solution is phosphate buffered saline.

52. The kit of claim 43 further comprising a third container, the third container containing an antiemetic agent or a hematopoietic colony stimulating factor.

53. The kit of claim 43 further comprising means for administering the liposomal platinum complex and capecitabine or a pharmaceutically acceptable salt thereof, to a subject.
